(19) United States
(12) Patent Application Publication
(10) Pub. No.: US 2020/0411187 A1
(43) Pub. Date: Dec. 31, 2020

(54) System And Method For Creating Synthetic And/Or Semi-Synthetic Database For Machine Learing Tasks

(71) Applicant: MEDECIDE LTD., Tel-Aviv (IL)

(72) Inventors: Josef Elidan, Tel-Aviv (IL); Orly Elidan-Harel, Hod-HaSharon (IL); Dan Berachowitz, Haifa (IL)

(21) Appl. No.: 16/976,828

(22) PCT Filed: Feb. 28, 2019
(86) PCT No.: PCT/IB2019/051609

§ 371 Date: Aug. 31, 2020

Related U.S. Application Data
(60) Provisional application No. 62/638,331, filed on Mar. 5, 2018.

(51) Int. Cl. G16H 50/20; G16H 10/60; G16H 40/20; G06F 40/20

(52) U.S. Cl. CPC G16H 50/20; G06F 40/20; G16H 40/20; G16H 10/60

(57) ABSTRACT

An automated method of creating synthetic and/or semi-synthetic medical files database for machine learning tasks, comprising: retrieving medical data from external sources; extracting information from the medical data; generating at least one first scenario comprising a plurality of medical factors using the medical data and a rules engine; receiving at least one contradiction marking; updating the rules engine; generating at least one second scenario comprising a plurality of medical factors using the medical data and the updated rules engine; and determining at least one medical procedure recommendation according to the at least one second scenario.

### CROSS-REFERENCE TO RELATED PATENT APPLICATIONS

This patent application claims priority from and is related to U.S. Provisional Patent Application Ser. No. 62/638,331, filed 5 Mar. 2018, this U.S. Provisional Patent Application incorporated by reference in its entirety herein.

### FIELD OF THE INVENTION

The present invention generally relates to the field of machine learning and specifically to a system and a method for creating synthetic and/or semi-synthetic medical cases and training datasets for machine learning tasks.

### BACKGROUND

In various fields of information science, attaining relevant information from the training data is crucial for machine learning tasks. However, in many cases this information lacks critical factors.

Supervised learning is the machine learning task of inferring a function from labeled training data. The training data consists a set of training examples, while each example is a pair consisting an input object (typically a vector) and a desired output value (also called the supervisory signal). A supervised learning algorithm analyzes the training data and produces an inferred function, which can be used for mapping new examples. An optimal scenario will allow the algorithm to correctly determine the class labels for unseen instances. This requires the learning algorithm to generalize from the training data to unseen situations in a “reasonable” way.

In many fields, such as medicine, a shortage of complete computed medical files with structured data is obstructing the machine learning process, which requires a large number of labeled structured training datasets.

Therefore, there is a need for a system and method for creating synthetic and/or semi-synthetic medical database comprising abundant synthetic or semi-synthetic medical files to be used in various fields of interest for various uses.

### SUMMARY

According to an aspect of the present invention there is provided an automated method of creating synthetic and/or semi-synthetic medical files database for machine learning tasks, comprising: retrieving medical data from external sources; extracting information from the medical data; generating at least one first scenario comprising a plurality of medical factors using the medical data and a rules engine; receiving at least one contradiction marking; updating the rules engine; generating at least one second scenario comprising a plurality of medical factors using the medical data and the updated rules engine; and determining at least one medical procedure recommendation according to the at least one second scenario.

The medical data may comprise at least one of patient's medical file, reports and free text notations.

According to another aspect of the present invention there is provided a computerized system for creating a synthetic and/or semi-synthetic medical files database for machine learning tasks, comprising: a rules engine; a system server configured to: communicate with structured and unstructured external medical sources; extract and store medical information from the external medical sources in a database; analyze the medical information; generate at least one scenario comprising a plurality of medical questions and answers using the medical information and the rules engine; receive at least one contradiction marking; update the rules engine; generate at least one scenario comprising a plurality of medical questions and answers using the medical information and the updated rules engine; and receive at least one recommendation; the system server comprising: a data mining and Natural Language Processing (NLP) module; a machine learning module; an Application Program Interface (API) module; at least one database; a web application configured to provide users with an interactive platform for communicating with the system; and a processing engine.

The medical data may comprise at least one of patient's medical file, reports and free text notations.

### BRIEF DESCRIPTION OF THE DRAWINGS

For a better understanding of the invention and to show how the same may be carried into effect, a reference will be made, purely by a way of example, to the accompanying drawings.

With a specific reference to the drawings in detail, it is stressed that the particulars shown, are by a way of example and for purposes of illustrative discussion of the preferred embodiments of the present invention only, and are presented in the cause of providing what is believed to be the most useful and readily understood description of the principles and conceptual aspects of the invention. In this regard, no attempt is made to show the structural details of the invention in more detail than is necessary for a fundamental understanding of the invention, the description taken with the drawings making apparent to those skilled in the art how the several forms of the invention may be embodied in practice. In the accompanying drawings:

FIG. 1 is a schematic block diagram of the system, according to embodiments of the present invention;

FIGS. 2A-2E shows an exemplary synthetic or semi-synthetic file (case, scenario) generated by the system of the present invention;

FIG. 3 shows an exemplary synthetic or semi-synthetic file (case, scenario) generated by the system of the present invention and marked by an expert;

FIG. 3A shows the selected conflicts which are saved by the system of the present invention in order to eliminate appearance of these combinations in future scenarios;

FIG. 4 shows another exemplary synthetic or semi-synthetic file (case, scenario) generated by the system of the present invention and marked by an expert;

FIG. 4A shows another exemplary synthetic or semi-synthetic file (case, scenario) generated by the system of the present invention and marked by an expert;

FIG. 5 shows an exemplary decision;

FIG. 6 is a flowchart showing an exemplary process performed by the system of the present invention;

FIG. 7 is a flowchart showing an exemplary process performed by the system of the present invention, after the generated files have minimal to no contradictions;

FIG. 8 shows an exemplary question in the rules engine;

FIG. 8A shows another exemplary question in the rules engine; and

FIG. 8B shows yet another exemplary question in the rules engine.

### DETAILED DESCRIPTION OF PREFERRED EMBODIMENTS

Before explaining at least one embodiment of the invention in detail, it is to be understood that the invention is not necessarily limited in its application to the details of the construction and the arrangement of the components and/or methods set forth in the following description and/or illustrated in the drawings and/or the Examples. The invention is capable of other embodiments or of being practiced or carried out in various ways.

As it will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire line, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on a remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The present invention provides a system and method for creating synthetic and/or semi-synthetic database to be used in various fields of interest for various uses such as, for example, as labeling a training data for machine learning tasks in the medical field. The system and method of the present invention may create large number of diverse synthetic and/or semi-synthetic medical files which may be improved over time.

It will be appreciated that throughout the specification hereinbelow the synthetic and/or semi-synthetic files may be referred as cases or scenarios.

At the first stage, by marking conflicts between features within the synthetic and/or semi-synthetic files and setting probability of their occurrence, the system may learn how to create optimized cases in the future, according to the specific issue (e.g., disease).

At the second stage, by labeling the cases, the system is able to learn how to better calculate the defined variables, and improve the obtained conclusions throughout time.

Machine learning systems often require a large training dataset in order to output results in high accuracy. One of the largest issues in the medical field is the lack of sufficient amount of training data to be provided to those systems.

Nowadays, the medical information within medical files is not fully documented and/or structured and therefore unsuitable for data processing by machine learning.

The system and method of the present invention allow examining a large number of diverse synthetic medical files as testing data, for validating the accuracy of the machine learning models.

The system creates diverse files which may include real data and synthetic data. The creation of the synthetic and/or semi-synthetic files is made based on labeling generated cases, with marking conflicts.

According to embodiments of the present invention, after a synthetic and/or semi-synthetic file is generated, it is presented to an expert (e.g., physician). The expert examines the file's data, validates that the parameters are relevant to the medical case and that there are no contradictions between the parameters.

According to embodiments of the present invention, the expert (physician) may mark the probability of different parameters to be presented in a file (percentage).

Every file inspected by the expert is saved by the system in order to enable the system to learn and create improved and more realistic files in the future.

It will be appreciated that the system of the present invention is not limited to saving all the files.

According to embodiments of the present invention, when the system generates a file with minimal to no contradictions, an expert (e.g., physician, the same or different from the first physician) may decide whether the file justifies a procedure (e.g., a medical operation) by labeling the case.

The algorithms described here are suitable for any machine learning domain, in particular but not limited to the medical field. Therefore, every system having machine learning capabilities may use this algorithm in order to create vast amount of training and testing data, needed for the learning process.

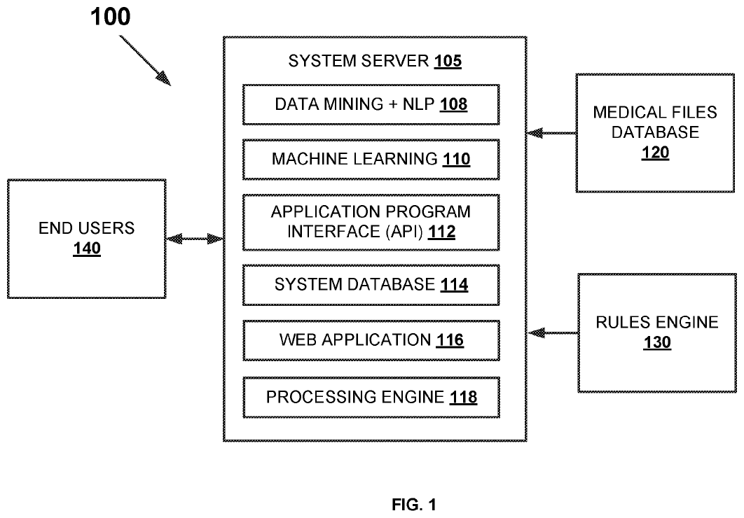

FIG. 1 is a schematic block diagram of the system according to embodiments of the present invention.

System 100 comprises one or more system servers (only one is shown) 105 communicating with at least one medical files database (only one is shown) 120, such as, for example, medical institutions' databases comprising patients' files; with rules engine 130 and with end users' electronic communication means 140, such as medical institutions' systems, patients' computers and/or mobile electronic communication devices. According to embodiments of the present invention, the end user may be an expert (physician).

System server 105 comprises a processor and some or all of the following computerized modules:
A data mining and Natural Language Processing (NLP) module 108, configured to extract information from medical files database(s) 120 and transform it into an understandable structure for further use, using NLP techniques. Data extracted includes, for example, data from patients' medical files such as lab reports, free text notations etc. The extracted data is used for automatically adding real features to the synthetic or semi-synthetic file.
A machine learning module 110, configured to:
   Calibrate the weight (impact) of each parameter relevant to each medical procedure, by analyzing a large number of scenarios.
   Calibrate the system using information mined from real medical files.
   Calibrate the system using expert's feedback.
   Calibrate the system by scanning latest researches, statistics and publications by health organizations (e.g., American Academy Guidelines, World Health Organization, American and European health organizations, etc.).
An Application Program Interface (API) module 112 configured to enable data retrieval from various external medical sources.
One or more synthetic and/or semi-synthetic database 114, storing the synthetic and/or semi-synthetic files.
A web application 116, providing users (e.g., experts) with an interactive platform for communicating with the system over the Internet, including presenting queries, receiving answers and receiving decisions.
A processing engine 118, configured to:
   Select and present a file including a plurality of parameters to the user (e.g., expert);
   Grade user's response according to contradictions markings and optionally percentage markings;
   Adjust the next file based on previous response(s).

The rules engine 130 comprises:
A set of parameters related to each medical condition (disease) derived, for example, from patients' medical files, statistics and guidelines of the American and European Academies, Japanese, or any other similar organization which may be changed by experts during the process;
A set of probabilities (percentage) associated with each parameter and represent the typical probability of a particular parameter to be presented. These probabilities are pre-determined according to general knowledge and may continuously be updated by experts.
A set of contradictions between parameters, which are pre-determined according to general knowledge and continuously updated by, for example, the latest research, statistics and guidelines of the American and European Academies, Japanese, etc. and by experts;

Typically, in a set-up phase, a set of contradictions for each medical/surgical procedure are generated in advance and saved in the rules engine, e.g., by human experts.

At the end of the process the system may automatically generate synthetic and/or semi-synthetic medical file, and may have the option to label the case whether a procedure is justified or not.

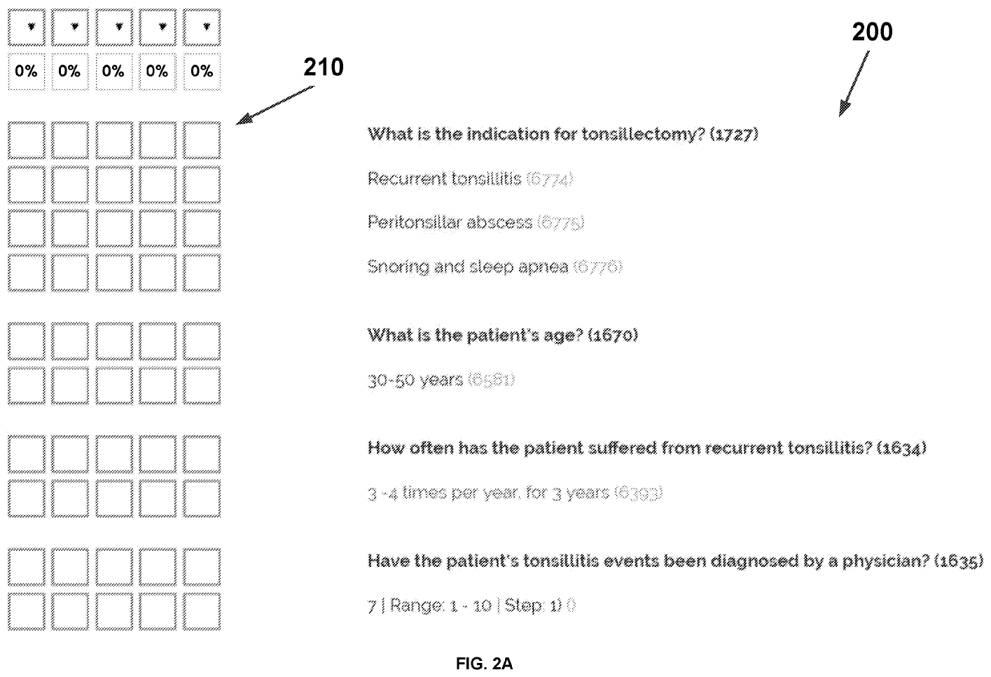

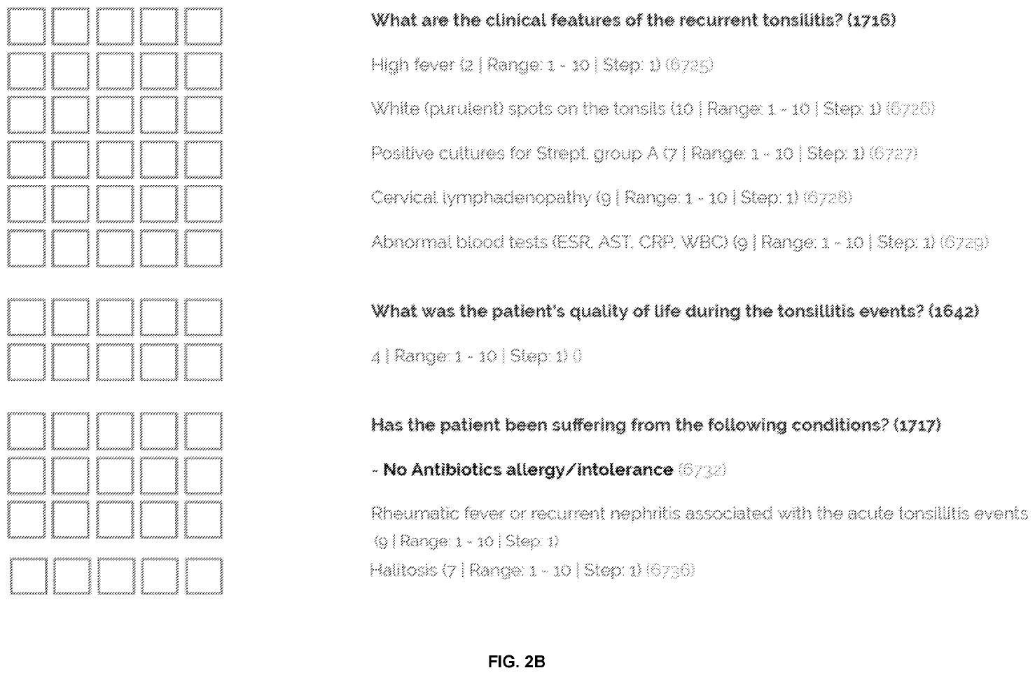

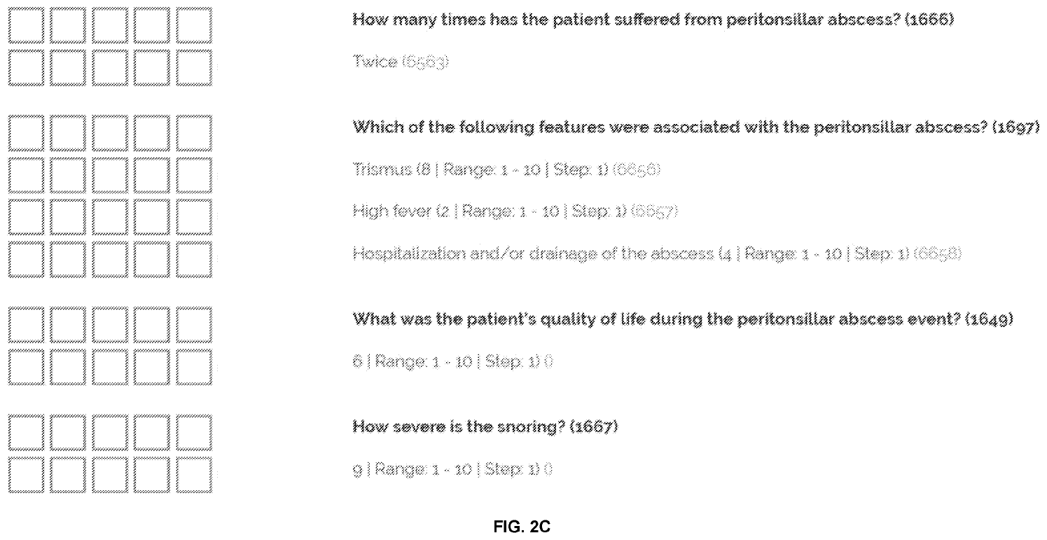

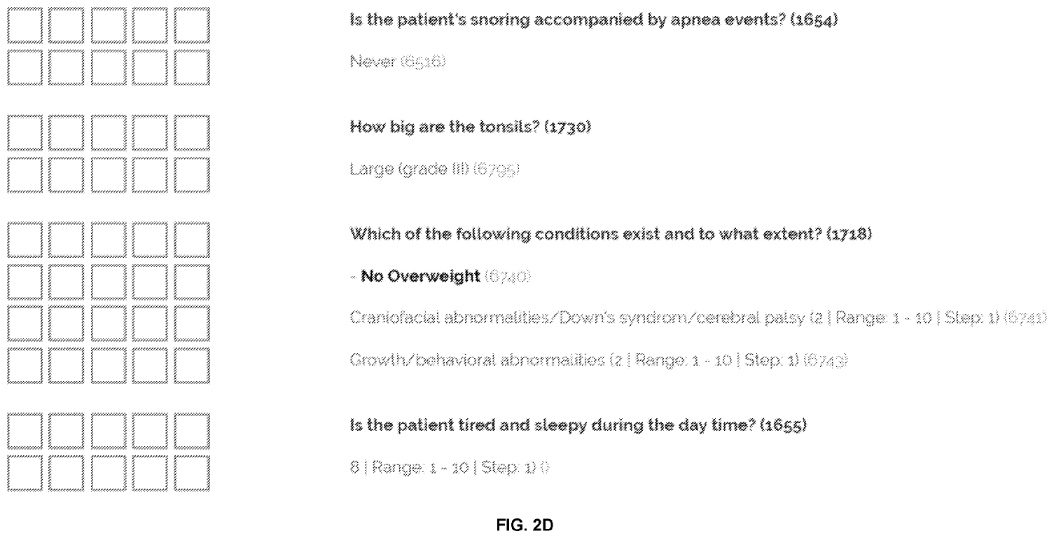

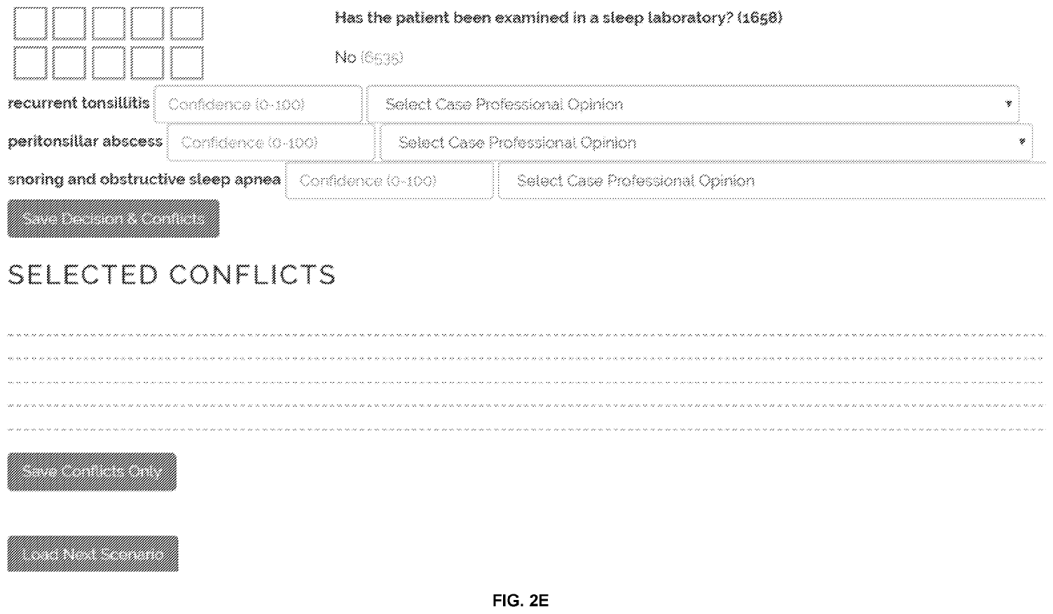

FIGS. 2A-2E shows an exemplary synthetic or semi-synthetic file (case, scenario) 200 generated by the system of the present invention.

The scenario comprises a list of medical factors according to the issue and the medical condition of a patient.

When the expert receives the scenario he may mark contradictions between factors using an easy to use user interface, such as square shaped checkboxes 210.

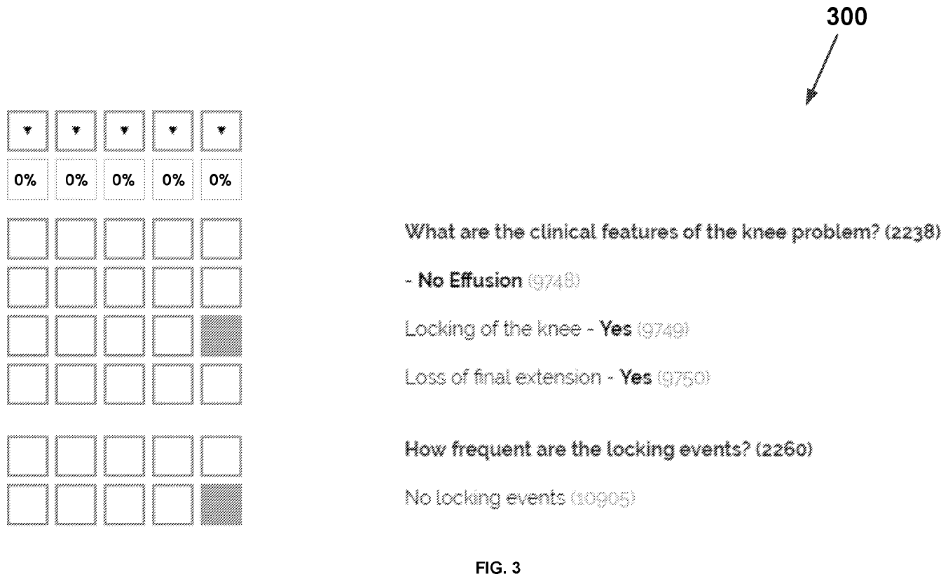

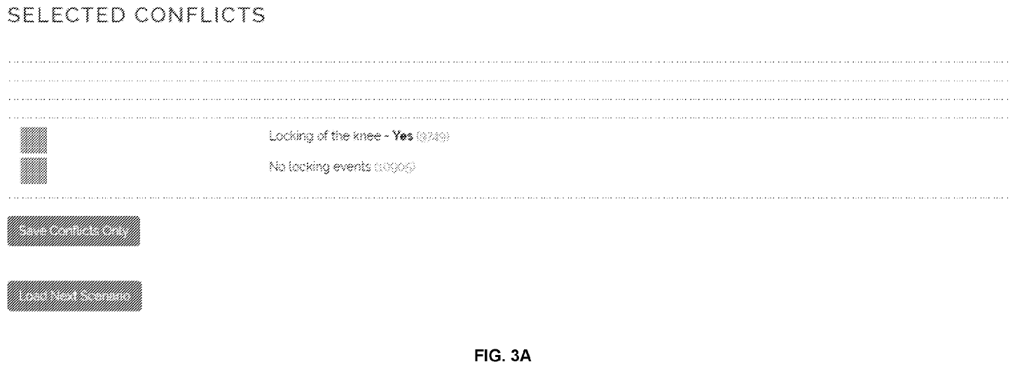

FIG. 3 shows an exemplary synthetic or semi-synthetic file (case, scenario) 300 generated by the system of the present invention and marked by an expert.

The scenario 300 comprises contradictions marked by an expert.

For example, there is no way (0% chance) that the answer “Yes” to the question “locking of the knee” can coexist with “No locking events”. The result and meaning of this “contradiction” marking is that in the next random scenarios there will be a 0% chance that these two answer appear synchronously

FIG. 3A shows the selected conflicts which are saved by the system of the present invention in order to eliminate appearance of these combinations in future scenarios.

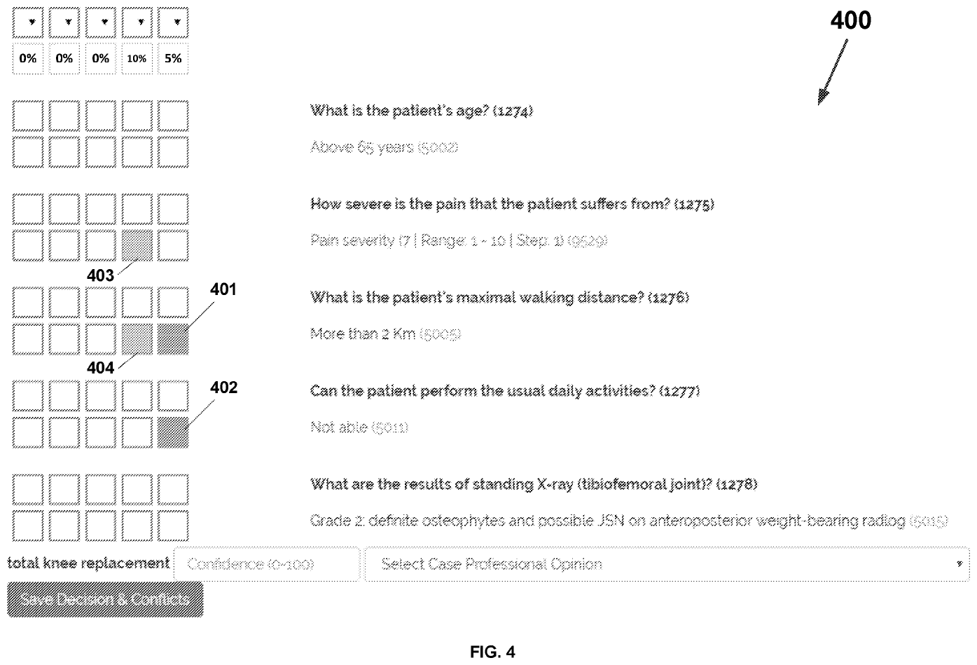

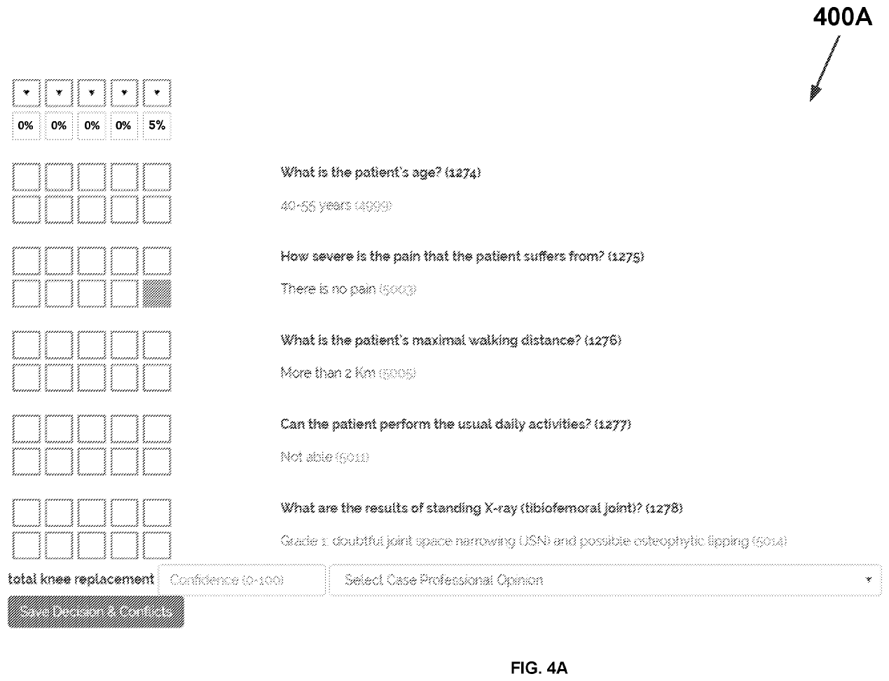

FIG. 4 shows another exemplary synthetic or semi-synthetic file (case, scenario) 400 generated by the system of the present invention and marked by an expert.

According to embodiments of the present invention, the scenario 400 comprises contradictions and probabilities (in percents) marked by an expert.

For example, the system would generate randomly only in 5% of the synthetic cases that variables 401 and 402 appear together.

In another example, the system would create randomly only in 10% of the synthetic cases that variables 403 and 404 appear together.

The selected conflicts are saved by the system of the present invention in order to apply to future scenarios according to the selected probability.

According to embodiments of the present invention, the system may enable an expert to determine upper and lower limits for forcing a range of conflicts, instead of a single conflict.

For example, if there is a low probability that a peritonsillar abscess shall occur until the age of 4 years and above the age of 80 years, the expert may choose four years as the lower limit and 80 years as the upper limit of age that would appear in the synthetic cases.

In another example, if there is no probability (0% chance) that a ninety years old patient exercises three times a week, the expert may choose ninety years as the limit, namely, there is no probability (0% chance) that 90-120 years old patients exercise three times a week.

FIG. 4A shows yet another exemplary synthetic or semi-synthetic file (case, scenario) 400A generated by the system of the present invention and marked by an expert.

This example demonstrates the adjustment of the probability (percentage) of a single variable (parameter).

The probability of the answer “There is no pain” was adjusted to 5%, i.e. this variable shall appear only in 5% of the future cases (scenarios).

To label a scenario, the expert marks the proper recommendation at the end of the questionnaire and a level of confidence of the decision.

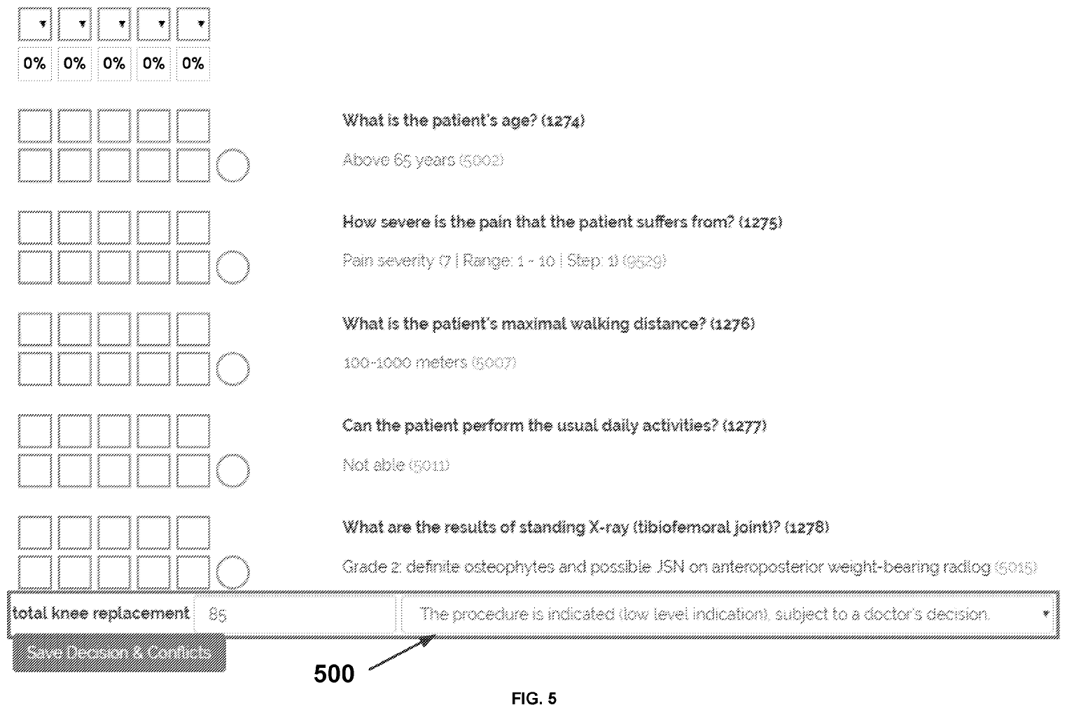

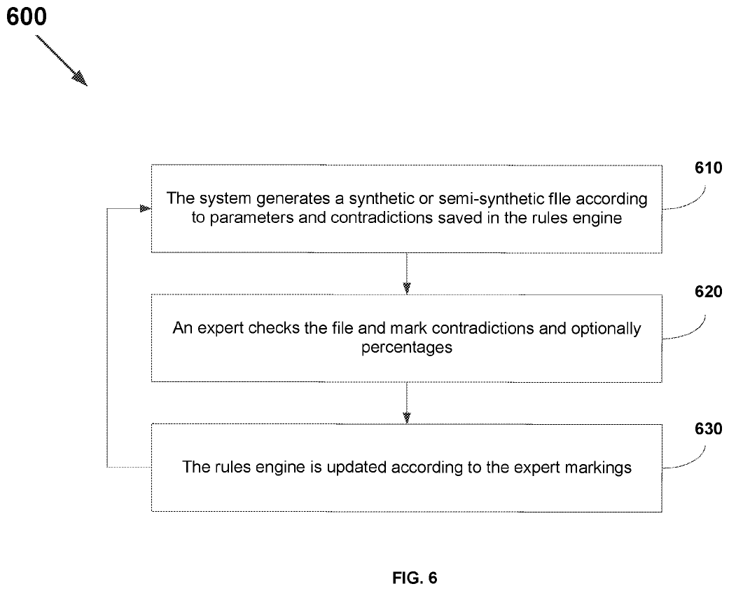

FIG. 5 shows an exemplary decision 500.

In the example of FIG. 5, the label is “The procedure is indicated (low level indication)” with a confidence level of 85%.

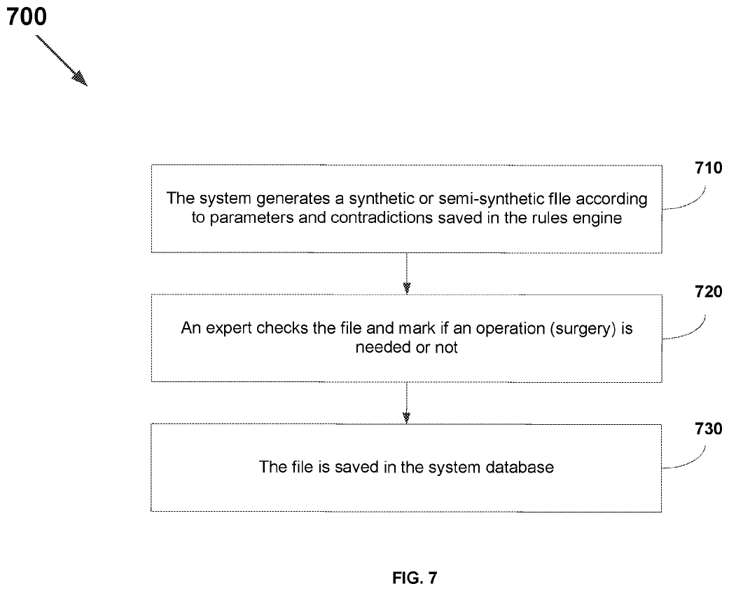

FIG. 6 is a flowchart 600 showing an exemplary process performed by the system of the present invention.

In step 610, the system generates a synthetic or semi-synthetic file according to parameters, rules and contradictions saved in the rules engine.

In step 620, an expert checks the file and marks full contradictions between the answers (0% chance of future appearance) or optionally percentages.

In step 630, the rules engine is updated according to the expert's markings.

The process then may return to step 610 up to a point where the generated files have minimal to no contradictions.

FIG. 7 is a flowchart 700 showing an exemplary process performed by the system of the present invention after the generated files have minimal to no contradictions.

In step 710, the system generates a synthetic or semi-synthetic file according to parameters, rules and contradictions saved in the rules engine.

In step 720, an expert checks the file and mark if a medical procedure is indicated (appropriate), not indicated (not appropriate), or the case needs further consideration (equivocal).

In step 730, the file is saved in the system database.

As a result of the processes described in conjunction with FIG. 6 and FIG. 7, the system may automatically generate a vast number of random synthetic and/or semi-synthetic files.

As mentioned above, in a set-up phase, a set of specific parameters, rules and contradictions for each medical/surgical procedure are generated, e.g., by human experts, in advance and saved in the rules engine.

Furthermore, these parameters, rules and contradictions may be updated according to updates in the research, statistics and new guidelines.

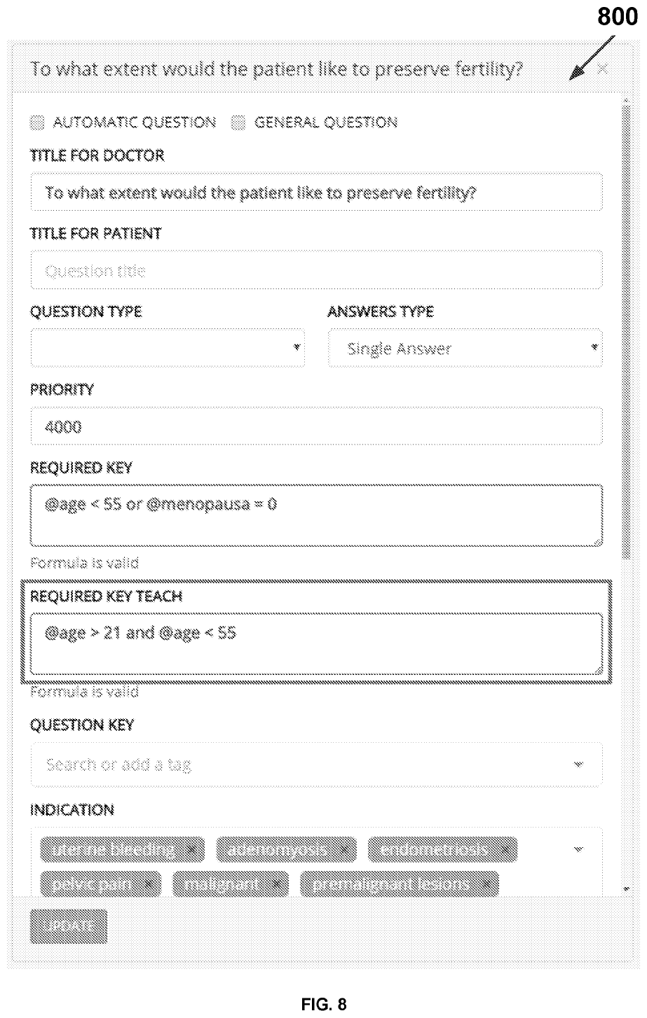

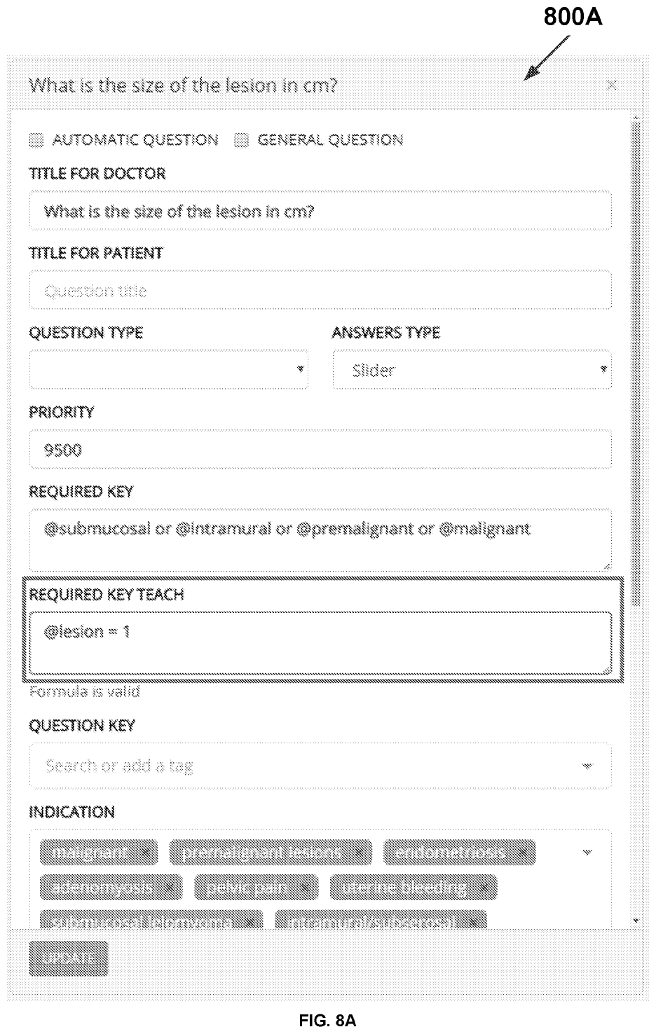

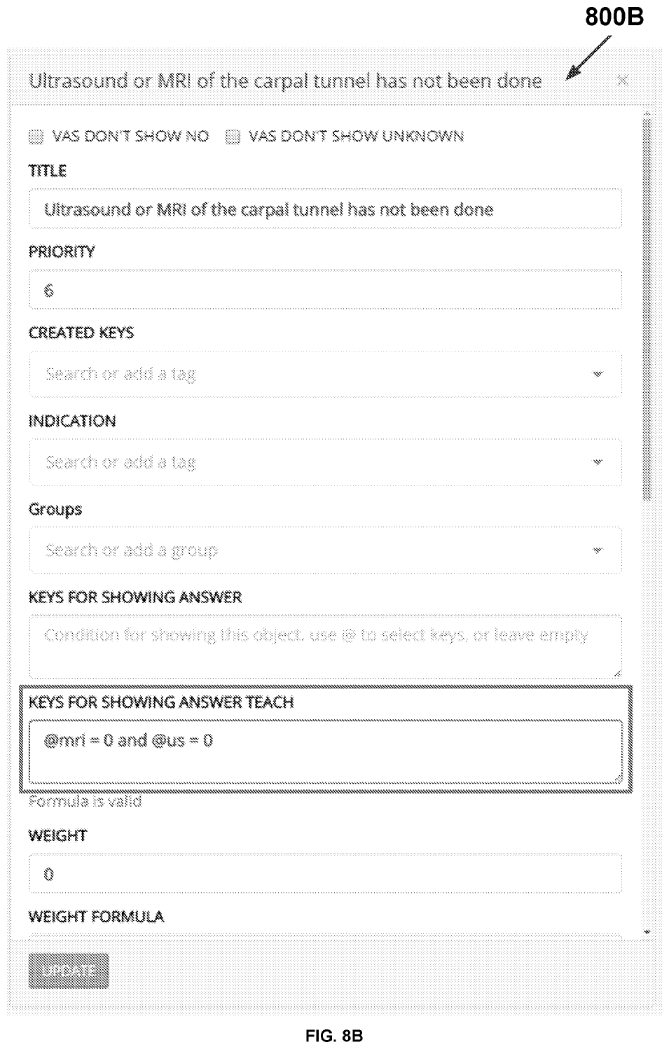

FIG. 8 shows an exemplary question 800 in the rules engine.

This example demonstrates the definition of a condition for the appearance of a question.

For the question “To what extent would the patient like to preserve fertility?” the patient's age must be in the range of 21-55.

FIG. 8A shows another exemplary question 800A in the rules engine.

A prerequisite for the question “What is the size of the lesion in cm?” to appear, the key @lesion=1 (which confirms the presence of a lesion) must be present.

FIG. 8B shows yet another exemplary question 800B in the rules engine.

This example demonstrates the definition of a condition for the appearance of a certain answer.

Similar to creating a condition for questions to appear, in this example for the answer “US or MRI of the carpal tunnel had not been done” to appear, certain conditions (keys) must have zero value (meaning: are not present). The condition states that this answer can only appear in a case if either the MRI test or the US test (or both) have not been performed.

It will be appreciated by persons skilled in the art that the present invention is not limited to what has been particularly shown and described hereinabove. Rather the scope of the present invention is defined by the appended claims and includes combinations and sub-combinations of the various features described hereinabove as well as variations and modifications thereof which would occur to persons skilled in the art upon reading the foregoing description.

1. An automated method of creating synthetic and/or semi-synthetic medical files database for machine learning tasks, comprising:
retrieving medical data from external sources;
extracting information from said medical data;
generating at least one first scenario comprising a plurality of medical factors using said medical data and a rules engine;
receiving at least one contradiction marking;
updating said rules engine;
generating at least one second scenario comprising a plurality of medical factors using said medical data and said updated rules engine; and
determining at least one medical procedure recommendation according to said at least one second scenario.

2. The method of claim 1, wherein said medical data comprise at least one of patient's medical file, reports and free text notations.

3. A computerized system for creating a synthetic and/or semi-synthetic medical files database for machine learning tasks, comprising:
a rules engine;
a system server configured to:
communicate with structured and unstructured external medical sources;
extract and store medical information from said external medical sources in a database;
analyze said medical information;
generate at least one scenario comprising a plurality of medical questions and answers using said medical information and said rules engine;
receive at least one contradiction marking;
update said rules engine;
generate at least one scenario comprising a plurality of medical questions and answers using said medical information and said updated rules engine; and
receive at least one recommendation;
said system server comprising:
a data mining and Natural Language Processing (NLP) module;
a machine learning module;
an Application Program Interface (API) module;
at least one database;
a web application configured to provide users with an interactive platform for communicating with the system; and
a processing engine.

4. The system of claim 3, wherein said medical data comprise at least one of patient's medical file, reports and free text notations.
